(19) United States
(12) Patent Application Publication
(10) Pub. No.: US 2022/0047654 A1
(43) Pub. Date: Feb. 17, 2022

(54) PHARMABIOTIC TREATMENTS FOR METABOLIC DISORDERS

(71) Applicant: UNIVERSITY OF SOUTH CAROLINA, COLUMBIA, SC (US)

(72) Inventors: MICHAEL D. WYATT, COLUMBIA, SC (US); CHLOE LEBEGUE, COLUMBIA, SC (US); JACOB MASSEY, COLUMBIA, SC (US)

(21) Appl. No.: 17/415,976

(22) PCT Filed: Dec. 18, 2019
(86) PCT No.: PCT/US2019/067093

§ 371 Date: Jun. 18, 2021

Related U.S. Application Data
(60) Provisional application No. 62/782,675, filed on Dec. 20, 2018.

(51) Int. Cl. A61K 35/747; A61K 9/00; A61K 31/519; A61K 38/44; C12N 9/02

(52) U.S. Cl. CPC A61K 35/747; A61K 9/0056; C12Y 114/16001; A61K 38/44; C12N 9/0071; A61K 31/519

(57) ABSTRACT

Herein are described pharmabiotic compositions and methods of treatment using genetically modified bacteria that include a portion or a variant of human cDNA sequence. Generally, the modified bacterium has a genetic modification that includes the introduction or inclusion of non-native DNA which contain a human cDNA sequence that can be propagated in the genetically altered bacterium. As an example, the non-native DNA can include one or more portions of human cDNA that encode the enzyme phenylalanine hydroxylase. In an embodiment, the modified bacterium can be provided to a patient as a treatment for a metabolic disorder. In a non-limiting example, a modified bacterium including human cDNA encoding the enzyme phenylalanine hydroxylase can be provided to a patient suffering from a deficiency in phenylalanine hydroxylase or suffering from a mutation to the native gene that results in an inactive form of the enzyme. As such, certain embodiments may provide methods for treating phenylketonuria using the modified bacterium.

### CROSS REFERENCE TO RELATED APPLICATION

This application claims filing benefit of U.S. Provisional Patent Application Ser. No. 62/782,675, having a filing date of Dec. 20, 2018, which is incorporated herein by reference for all purposes.

### BACKGROUND

Metabolic disorders, such as phenylalanine hydroxylase deficiency, affect a large portion of the US population. These disorders can negatively impact the health and well-being of patients diagnosed with a disorder starting as early as birth. In the case of phenylalanine hydroxylase deficiency, the disorder is an autosomal recessive genetic condition that affects 1 of every 15,000 infants born in the United States. Phenylalanine hydroxylase is an intrinsically hepatic enzyme that is responsible for the breakdown of the essential dietary amino acid phenylalanine into tyrosine. In cases of phenylalanine hydroxylase deficiency (also known as phenylketonuria), the enzyme is either nonfunctional or partially functional, secondary to a mutation in the phenylalanine hydroxylase gene (PAH). Deficient expression of phenylalanine hydroxylase results in supraphysiologic plasma levels of phenylalanine upon consumption of foods containing phenylalanine. Phenylalanine is present in dietary sources of protein such as fish, meat, nuts and eggs. Diagnosis is made primarily from plasma screenings of newborns. Such screenings were made mandatory in the United States in the 1960s. Hyperphenylalaninemia is diagnosed when untreated blood levels of phenylalanine are greater than the population norm of 0.06-0.1 mmol/L but less than the 1.2 mmol/L, diagnostic of classical phenylketonuria. Untreated classical phenylketonuria is associated with the most severe manifestations of the metabolic disorder.

The management of metabolic disorders like phenylalanine hydroxylase deficiency poses a challenge not only for healthcare providers, but also for the families of those affected. A 2016 cross-sectional study performed in the United Kingdom reported that a median of 19 hours per week was spent by caretakers in activities related to the management of the disorder. According to the National PKU Alliance of the United States, treatment of phenylalanine hydroxylase deficiency costs approximately $15,000 per year. Third-party payer support is payer-specific and variable. The alliance also states that inpatient care for a patient who has sustained neurological damage secondary to phenylketonuria may cost upwards of $200,000 per year. It can be inferred that the burden of this cost would fall upon the U.S. healthcare system.

Phenylalanine hydroxylase deficiency is touted as the textbook example of metabolic disorders without cure. However, the psychosocial, physical, and financial ramifications of phenylketonuria necessitate that treatments extending beyond the current standards of care be explored. Concomitantly, the human microbiome is one of the most rapidly advancing fields of research today. As research into the microbiome continues to produce various biome-altering formulations, it is inevitable that numerous applications will be discovered for such products, providing improvements and alternatives to current standards of care for metabolic disorders.

### SUMMARY

Embodiments of the disclosure are directed to genetically altered organisms and methods of using the genetically altered organisms as pharmabiotic treatments. In an embodiment, DNA constructs which include human cDNA can be introduced and propagated in microorganisms, including microorganisms of the genus Lactobacillus and Escherichia, to produce a modified bacterium. In an example embodiment, these DNA constructs can include one or more portions of human cDNA that encode an enzyme (e.g., phenylalanine hydroxylase). In another embodiment, the modified bacterium can be provided to a patient as a treatment for a metabolic disorder. For example, a modified bacterium encoding the enzyme phenylalanine hydroxylase can be provided to a patient suffering from a deficiency in phenylalanine hydroxylase or suffering from a mutation to the native gene that results in an inactive or partially active form of the enzyme. As such, certain embodiments can provide methods for treating phenylketonuria by delivering a modified bacterium to a patient.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIGS. 1A and 1B illustrate gel images as supported by embodiments of the disclosure.

FIGS. 2A and 2B illustrate gel images as supported by embodiments of the disclosure.

FIG. 3 illustrates an image of a gel as supported by embodiments of the disclosure.

FIG. 4 illustrates an image of a gel as supported by embodiments of the disclosure.

FIG. 5 illustrates an image of a gel as supported by embodiments of the disclosure.

FIG. 6 illustrates an image of a gel as supported by embodiments of the disclosure.

FIG. 7 illustrates a sequence comparison of a query sequence (SEQ ID NO: 4) with a subject sequence (SEQ ID NO: 5) as supported by embodiments of the disclosure.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, embodiments disclosed herein are directed to genetically altered organisms and methods using the genetically altered organisms. In an embodiment, DNA constructs which include human cDNA can be introduced and propagated in microorganisms, including microorganisms of the genus Lactobacillus and Escherichia, to produce a modified bacterium. In an example embodiment, these DNA constructs can include one or more portions of human cDNA that encode the enzyme phenylalanine hydroxylase as described in SEQ ID NO: 1. In another embodiment, the modified bacterium can be provided to a patient as a treatment for a metabolic disorder. For example, a modified bacterium encoding the enzyme phenylalanine hydroxylase can be provided to a patient suffering from a deficiency in phenylalanine hydroxylase or suffering from a mutation to the native gene that results in an inactive or partially active form of the enzyme. As such, certain embodiments can provide methods for treating phenylketonuria or other metabolic diseases by delivering a modified bacterium to a patient.

An embodiment of the disclosure can include a modified bacterium of the genus Lactobacillus that includes a genetic modification. In these embodiments, the genetic modification can include the introduction of a human cDNA sequence, or portions or mutants thereof, encoding an enzyme that encourages conversion of phenylalanine to tyrosine. In certain embodiments, the human cDNA sequence can include SEQ ID NO: 2, SEQ ID NO: 3, SEQ ID NO: 4, SEQ ID NO: 5, or SEQ ID NO: 6. In some embodiments, a genetic modification can include introduction of a sequence as is known in the art encoding a phenylalanine hydroxylase to form a modified bacterium. Sequences known in the art may be found for example in the NCBI database, specific examples of which include NCBI Reference Sequence NM_000277.2 and NM_000277.3.

Portions or mutants of disclosed sequences are also considered within the scope of this disclosure, providing the portion or mutant encodes a polypeptide that retains desired enzyme activity. For example, mutants can include alterations to SEQ ID NOs: 2-6 that encode one or more amino acid substitutions to SEQ ID NO: 1 or a portion thereof (e.g., mutating a codon for valine to a codon for alanine). Additionally, or alternatively, mutants of a sequence introduced to an organism as described can include one or more point mutations to the native cDNA sequence to substitute a degenerate codon for a native codon.

For embodiments of the disclosure that include a mutant of a sequence as described, the mutant can include one or more codon mutations that modify the expressed protein to substitute one hydrophobic amino acid (e.g., valine) for another hydrophobic amino acid (e.g., alanine, leucine, isoleucine, proline, phenylalanine, methionine, or tryptophan) to produce an enzyme variant. Amino acids can be categorized as having hydrophobic, hydrophilic, and aromatic side chains. Embodiments of the disclosure can include a genetically modified bacterium that includes a mutant of a nucleotide sequence as described, the mutant encoding an enzyme variant. In these embodiments, the one or more substitutions can modify the native protein sequence (e.g., SEQ ID NO: 1) to substitute one amino acid for a second amino acid, where both have the same-side chain category (e.g., hydrophilic). Other possible side chain categories can include size and charge.

Due to codon redundancy, there are many theoretically possible cDNA sequence variants that could encode an enzyme such as phenylalanine hydroxylase. Additionally, enzyme variants that modify the native protein sequence, while retaining enzyme activity, further increase this number. Herein, embodiments of the disclosure can include a genetically modified organism having a genetic modification that includes the entirety of one of SEQ ID NOs: 2-6, a portion of one of SEQ ID NOs: 2-6, or a mutant of one of SEQ ID NOs: 2-6. For these embodiments, the genetic modification results in the expression of a protein (e.g., phenylalanine hydroxylase) or a protein variant or a partial protein that retains the function of the native protein or enzyme. Some exemplary mutations that would result in protein variants are described; however, these are not meant to limit the scope of mutations that can produce enzyme variants.

In embodiments of the disclosure, the modified bacterium can express an enzyme encoded in an introduced cDNA sequence. For example, a modified bacterium can be created that expresses the enzyme phenylalanine hydroxylase. In an embodiment, the modified bacterium including human cDNA can convert phenylalanine to tyrosine.

In some embodiments, the modified bacterium can include a second, third, or more genetic modifications. Several non-limiting examples of an additional genetic modification can include an antibiotic resistance sequence, a control sequence, or a monitoring sequence. For embodiments that include one or more additional genetic modifications, an additional genetic modification can be associated with the sequence that encodes a polypeptide that encourages conversion of phenylalanine to tyrosine such that genetic expression would result in a linked effect. As an example, the linked effect can be a control sequence, such as a promotor region, that can adjust gene expression of the human cDNA sequence. In certain embodiments, the promotor region can respond to a compound to increase expression of the cDNA sequence, thus increasing mRNA production and subsequent protein synthesis. As another example, the linked effect can be a monitoring sequence that can be detected or provide an expression product that can be detected when the human cDNA is expressed in the modified bacterium. As an example, the monitoring sequence can encode a fluorescent protein such as green fluorescent protein (GFP), and the monitoring sequence can be linked to the cDNA sequence to produce genetic expression of an enzyme linked to GFP. Thus, the expression of the human cDNA could be tracked by fluorescence measurements. As another example, the linked effect can produce an antibiotic resistance. In embodiments of the disclosure, these or other second genetic modifications can be incorporated alone or in combination.

In certain embodiments, the second genetic modification can include a sequence encoding a signal linked to the enzyme that can be used to detect a secreted form of the enzyme. In these embodiments, detection of the secreted form of the enzyme can indicate that following enzyme synthesis in the cytosol, the enzyme has been delivered to an area outside of the cell membrane of the transgenic organism.

Additionally, some embodiments of the modified bacterium may include a modification to the native genetic sequence of the bacterium.

Embodiments of the modified bacterium described herein can be capable of surviving in the human gut for at least 4 hours. Certain embodiments can be capable of surviving in the human gut for up to 24 hours. Thus, embodiments of the disclosure can provide a modified bacterium capable of surviving in the human gut for about 4 to about 24 hours.

In any of the embodiments of the disclosure, the modified bacterium of the genus Lactobacillus can be L. helveticus. In certain embodiments, the modified bacterium can include a combination of one or more species selected from the group: L. helveticus, L. acidophilus, L. salivarius, L. casei, L. curvatus, L. plantarum, L. sakei, L. brevis, L. buchneri, L. fermentum, and L. reuteri.

Embodiments of the disclosure can also provide methods for treating a metabolic disorder using various embodiments of the modified bacterium disclosed above. For example, an embodiment of the disclosure can include delivering a modified bacterium to a patient via one of several administration routes. In these embodiments, the administration route can include one or more pathways selected from, and without limitation to: oral, rectal, nasogastric, percutaneous gastronomy endoscopy (PEG) tube, nasoduodenal tube, and jejunostomy tube.

In an example embodiment, a method for treating a metabolic disorder can include delivering a modified bacterium encoding a cDNA for a phenylalanine hydroxylase to a patient diagnosed with phenylalanine hydroxylase deficiency (also known as phenylketonuria).

In some embodiments, the method for treating a metabolic disorder can include a dosage regimen. For these embodiments, the dosage regimen provides a schedule for delivering the modified bacterium. In an embodiment, the modified bacterium can be delivered daily. In another embodiment, the modified bacterium can be delivered at meals. For certain metabolic disorders, such as phenylalanine hydroxylase deficiency, there could be an advantage to using a dosage regime at meals, especially meals that include protein, due to the spike in phenylalanine blood concentration following protein digestion and adsorption. In some embodiments, the modified bacterium can be provided as part of a live culture. An example live culture can include delivering the modified bacterium as part of a yogurt, which would provide advantages for delivering the modified bacterium orally and with a meal. In some embodiments, the modified bacterium can be provided as a bacterial slurry, a lyophilized powder, or in various manifestations of microencapsulation or standard encapsulation.

Embodiments of the disclosure can provide methods of treatment for various patients. Herein, the term patient is not meant to be interpreted as a limitation. A patient can be a human or animal of any age group or gender unless specifically noted. Certain embodiments may include delivering a modified bacterium to a pregnant mother, and certain embodiments may include delivering a modified bacterium to a newborn child. In a newborn child, the gut has not been colonized by bacteria and in certain embodiments, a newborn (e.g., a child up to about 6 months of age) diagnosed with phenylketonuria can be treated with the modified bacterium substantially right after birth.

Some embodiments may include a secondary treatment, such as co-administering a drug and/or providing a pretreatment. For example, certain embodiments of the disclosure can include methods where a patient initially receives an antibiotic course to eliminate some bacteria from the gut before delivering the modified bacterium. In some embodiments, the patent may receive an antibiotic course during delivery of the modified bacterium.

As another example, pterin cofactors, such as, tetrahydrobiopterin, or other manufactured, exogenous pterin cofactors such as sapropterin can be provided as the secondary treatment before, during, or after delivery of the modified bacterium. Additionally, or alternatively, the secondary treatment can include a dietary restriction. In certain embodiments, the dietary restriction can include a low-phenylalanine or low protein diet.

Production of the modified bacterium and introduction of the human cDNA into the modified bacterium may be accomplished by a variety of methods. Exemplary methods are provided herein, but these are not meant to limit the scope of variations that are contemplated. Introduction of plasmids or alteration of the native genetic sequence in combination or alone can be used to produce the genetically altered bacterium. Provided herein is a non-limiting example demonstrating the introduction human cDNA to L. helveticus bacteria using a plasmid to transfer the non-native cDNA to the bacteria and produce a modified bacterium.

The present disclosure may be better understood with reference to the Methods and Results set forth below in combination with the Drawings.

### Example 1

Example 1 discusses various methods and provides exemplary embodiments that may be understood in conjunction with the Drawings and Description provided herein. The materials and conditions described in the example are demonstrative and are not meant to constrain the scope of the disclosure only to the materials and conditions used.

### Materials and Methods

### Propagation of Bacterial Strains

Escherichia coli were utilized to amplify DNA for manipulation. Specifically, stock strains of electrocompetent Escherichia coli cells were obtained from Lucigen. The plasmid pCMV6-XL4 containing the cDNA for human phenylalanine hydroxylase (PAH) was obtained from Origene. pCMV6-XL4 was introduced into E. coli by the preset E. coli protocol of the BioRad Gene Pulser Xcell™ Electroporation System. E. coli containing the plasmid pTRKH2 were purchased from Addgene and propagated. pTRKH2 is a well-known shuttle vector that can be propagated in both E. coli and gram-positive bacteria such as Lactobacillus helveticus. Wild-type L. helveticus strain number #15009 was purchased from ATCC.

E. coli were grown aerobically, agitated at speeds between 180 and 200 rpm, angled, and incubated overnight at 37° C. TB broth was used for growth in most instances, although no substantial variations were noted between the growth of E. coli in either LB or TB. MRS broth was used for propagation of L. helveticus, which were grown in anaerobic conditions without shaking at 37° C. L. helveticus was allowed 48 hours for growth on plates and 72 hours for growth in liquid culture.

### Plasmid Production

The pCMV6-XL4 and pTRKH2 plasmids were amplified by growing E. coli harboring the respective plasmids in TB plus appropriate antibiotic for selection. E. coli containing pCMV6-XL4 were propagated in 1-3 milliliters of liquid culture after loop inoculation of TB media containing 50 μg/ml ampicillin and incubated overnight at 37° C. with angled shaking at 180 rpm. Liquid cultures were grown for no less than sixteen hours. The gram-positive shuttle vector pTRKH2 was amplified by propagating E. coli as above but with the use of 150 μg/mL erythromycin for selection. Both plasmids were isolated from E. coli using a Qiagen MiniPrep plasmid extraction kit and following the manufacturer's protocol.

### Determination of Sample DNA Concentration

All sample DNA concentrations were determined in 2 μL aliquots via use of NanoDrop™ Spectrophotometer. Measurements were performed per manufacturer standards, including the use of appropriate blank solutions.

### Gel Electrophoresis, Restriction Enzyme Digests, Gel-Based Isolation of DNA Fragments

All gel electrophoresis was carried out on 0.8% agarose gels for identification of correct plasmid size and estimation of quality and purity. Restriction enzymes Not1, Sac1 (High-Fidelity®), and Sal1 were obtained from New England Biolabs. Digests were carried out per manufacturer instructions. To prepare for ligation of the PAH cDNA into the pTRKH2 backbone, the DNA samples were each incubated with both Sac1 and Sal1, followed by separate gel electrophoresis. The plugs of agarose gel containing each DNA fragment were excised from the gel with a razor blade. Digested DNA was purified from the agarose using a Thermo Scientific GeneJET Gel Extraction kit #K0691 protocol.

### Polymerase Chain Reaction (PCR)

PCR of the cDNA for phenylalanine hydroxylase was carried forward with 12.5 μL of Mastermix, 1.25 μL of forward and reverse primers at 10 μM each, 3 μL of the isolated Not1 fragment from pCMV6-XL4 at a concentration of 3 ng/μL and 7 μL of sterile DNAse and RNAse-free Nase-free water. Standard PCR reactions commonly involve an initial activation step, followed by three-step cycling of denaturation at 94° C., annealing at primer-specific temperatures, and extension at 72° C. for Taq polymerase. An annealing temperature of 57° C. was used for the primers designed for human PAH. The forward primer is:

(SEQ ID NO: 7)
5′-ATGTCCACTGCGGTCCTGGAAAACCCAGGCTTG

The reverse primer:

(SEQ ID NO: 8)
5′-TTACTTTATTTTCTGGAGGGCACTGCAAAGGATTCC

A second PCR reaction was performed as above to introduce Sac1 and Sal1 cut sites on the PAH isolate with a concentration of 3 ng/μL. The forward primer, including the additional sequence of the SAC1 cut site, was:

(SEQ ID NO: 9)
5′-CGCGGAGCTCATGTCCACTGCGGTCCTGGAAAACCCAGGCTTG

and the reverse primer, containing the SAL1 cut site was:

(SEQ ID NO: 10)
5′-GCGCGTCGACTTACTTTATTTTCTGGAGGGCACTGCAAAGGATTCC

### Ligation

DNA ligase was obtained from Lucigen. The ligation was performed per manufacturer instructions between the double-digested products of the shuttle vector pTRKH2 and the phenylalanine hydroxylase cDNA fragment that was amplified by PCR of the PAH cDNA fragment isolated from the pCMV6-XL4 plasmid. 4 μL of digested pTRKH2, 7 μL of the digested PAH fragment, 1 μL of DNA ligase, and 1.5 μL each of 10× ligation buffer and DNAse and RNAse-free water were placed together in a microcentrifuge tube and incubated at room temperature for five minutes. The tube was then incubated in a water bath at 70° C. for 15 minutes and then centrifuged for one minute at 10,000 rpm. A representative resulting plasmid was termed LiLi5 (SEQ ID NO: 11).

### Electroporation of E. Coli

Electroporation of E. Coli was performed per BioRad Gene Pulser Xcell™ Electroporation System preset protocol for E. Coli, as specified by manufacturer instructions.

Electroporation of Lactobacillus helveticus

Preparation of electrocompetent Lactobacillus helveticus followed the protocol described by Welker (doi:10.1093/femsle/fnu033). For the electroporation of the prepared Lactobacillus helveticus, 50 μL of electrocompetent cell suspension was loaded into electroporation microcuvettes followed by 200 ng of sample plasmid DNA. In separate cuvettes, pTRKH2, LiLi5 (SEQ ID NO: 11), and a control with no plasmid were subjected to an electroporation in a Bio-Rad Gene Pulser Xcell™ under the following conditions: 25 μF capacitance, 400Ω resistance, and 2000 V. After retrieval from the cuvette, 100 μL of each cell suspension was then placed in 900 μL of MRS recovery media and incubated for 4 hours at 37° C. The entire volume of the incubated cell suspensions was then transferred into 10 mL of MRS broth containing 0.5μ/mL erythromycin for antibiotic selection in liquid culture. A control of wild type Lactobacillus helveticus lacking plasmid was also incubated with 0.5 mcg/mL erythromycin to confirm positive selection in the presence of plasmids containing antibiotic resistance.

### Results

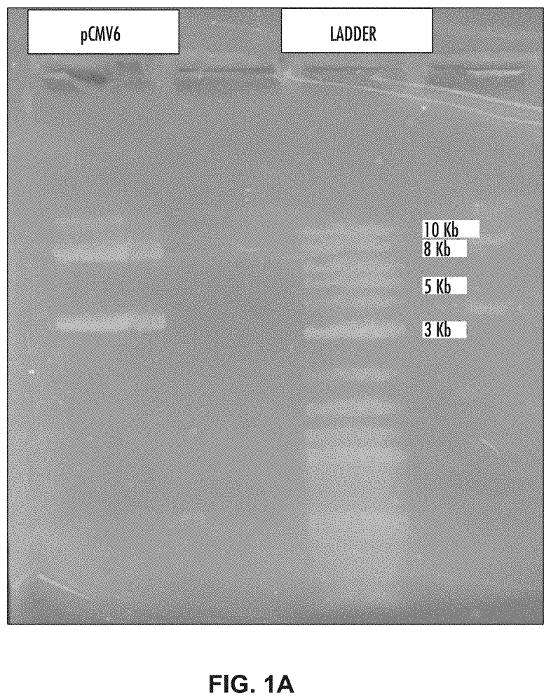

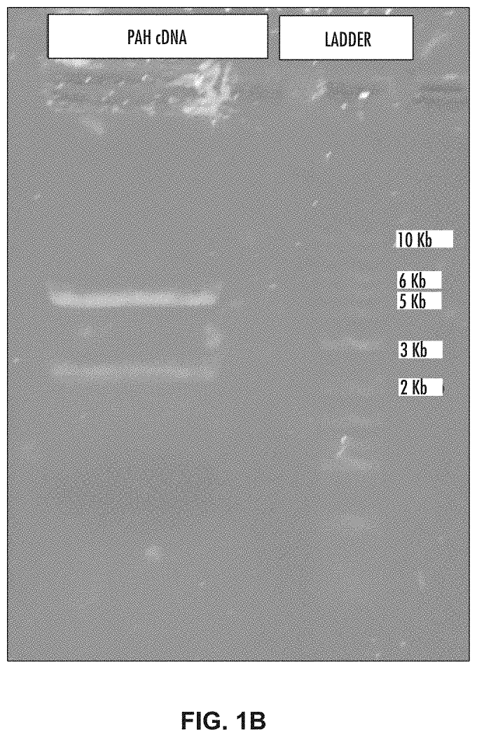

The first step in the process of creation of the genetically modified Lactobacillus helveticus was to isolate the plasmid containing the cDNA for the PAH enzyme. The far left lane of FIG. 1A characterizes pCMV6-XL4 DNA. The plasmid was identified by size comparison to a BioRad Log 2 Ladder, lane 3 of FIG. 1. This figure demonstrates that pCMV6-XL4 was successfully propagated in and subsequently isolated from E. coli. The appearance of two bands shows that the plasmid was visualized in two predominant forms, open circular and supercoiled.

The pCMV6-XL4 plasmid was next digested with the restriction enzyme Not1. The manufacturer's stated size of pCMV6-XL4 backbone was 4.7 kb with a 2.3 kb insert containing the PAH cDNA. Not1 sites flank the PAH cDNA, thus Not1 digest removes the cDNA from the backbone. FIG. 1B shows an agarose gel of the pCMV6-XL4 plasmid cut twice with Not1 in comparison to a log 2 ladder. This result confirms that a DNA fragment corresponding to the 2.3 kb fragment that contains the human PAH cDNA was successfully excised from the pCMV6-XL4 plasmid after Not1 digest.

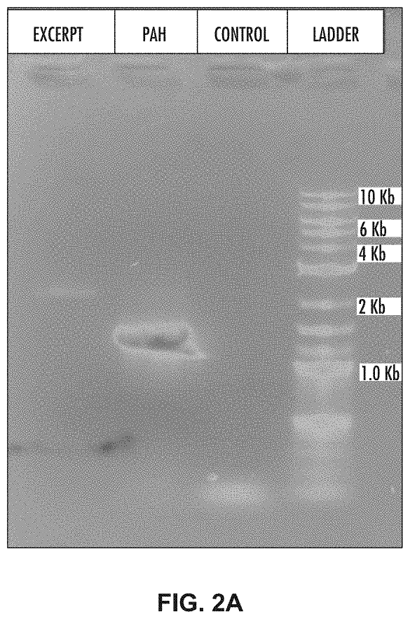

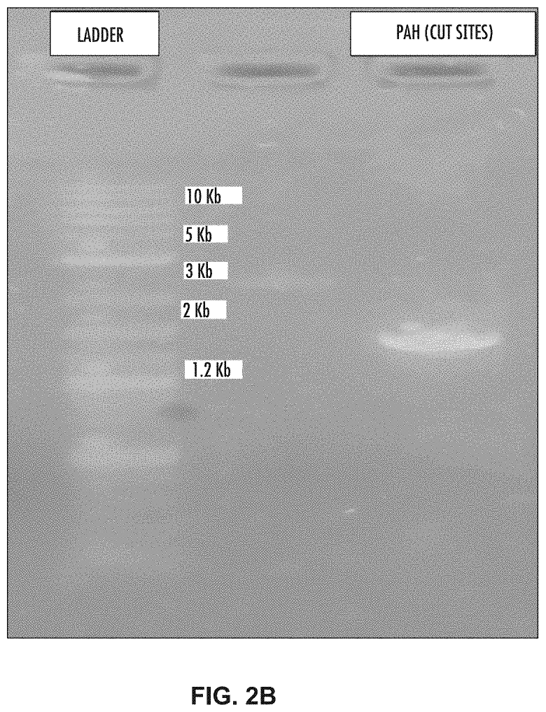

Next, PCR was used to specifically amplify the human PAH cDNA from the 2.3 kb fragment excised from the pCMV6-XL4 plasmid. Lane 1 of FIG. 2A shows the 2.3 kb fragment released from digesting the PCMV6-XL4 plasmid with Not1 and purified by gel electrophoresis. Lane 2 demonstrates the result of PCR performed on the Not1 fragment in lane 1 using primers specific to human PAH cDNA (SEQ ID NO: 7, SEQ ID NO: 8), yielding a 1.3 kB product (SEQ ID NO: 2). This result confirms that the primers specific to human PAH cDNA amplified a 1.3 kb portion of cDNA within the 2.3 kb insert removed from the pCVM6-XL4 plasmid. No amplification was seen with the control PCR, confirming that the reaction was specific to amplification of human PAH cDNA with the primers used. An additional PCR was performed using the same conditions as above, but with the substitution of primers containing Sac and Sal1 enzyme cut sites (SEQ ID NO: 9, SEQ ID NO: 10). Primers with additional flanking cut sites were used to provide compatible ends for subsequent ligation of the PAH cDNA into the pTRKH2 shuttle vector backbone (SEQ ID NO: 11).

FIG. 2B shows a gel run with 3 μL of digested PAH cDNA after PCR amplification in the far right lane using primers with the flanking enzyme cut sites.

The PCR product of PAH containing enzyme cut sites at the 5′- and 3′-ends was then subjected to a double restriction enzyme digest and loaded onto a gel to separate and purify the DNA fragment with ligatable ends (SEQ ID NO: 2). After electrophoresis, a small plug of agarose containing the DNA of interest was excised from the gel, and the DNA subsequently purified away from the agarose as described above.

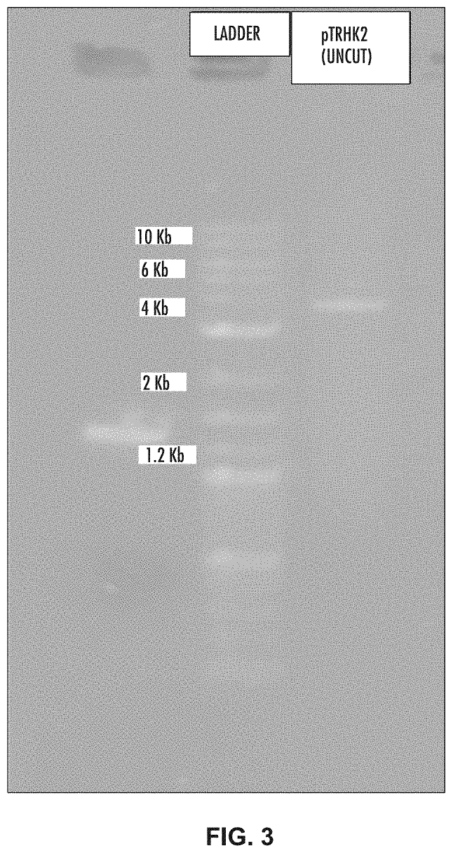

pTRKH2 was also isolated from E. coli and subjected to gel electrophoresis to confirm successful extraction. The right lane of FIG. 3 shows 5 μL of pTRKH2 DNA in comparison to a log 2 ladder. The left lane of FIG. 3 shows the same DNA as FIG. 2B for reference.

In preparation for ligation with the PAH cDNA fragment, pTRKH2 was double digested by Sac and Sal1 restriction enzymes, separated on a gel, and purified from the agarose plug as described above.

The ligation was then performed between the purified double-digest products of the shuttle vector pTRKH2 and the PAH cDNA fragment that was PCR amplified, yielding the PAH cDNA with flanking enzyme cut sites.

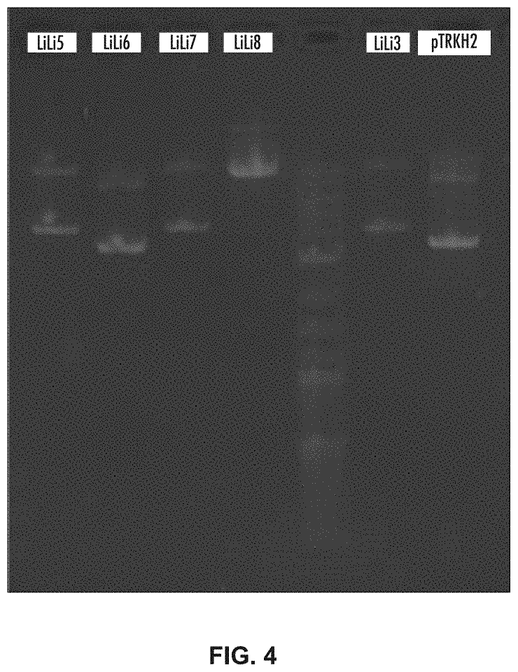

The products of the ligation reactions were electroporated into stock strains of E. coli. Native pTRKH2 plasmid and vehicle (no plasmid) also went through the electroporation protocol as controls. After electroporation, 15 and 150 μL aliquots of cells were plated on LB plates containing 150 mcg/ml erythromycin and incubated for 15 hours at 37° C. E. coli colony formation was noted for cells containing ligation products or pTRKH2, but not for E. coli lacking plasmid. Individual colonies from the pTRKH2-PAH ligation reactions were isolated and streaked out on a secondary plate and for overnight incubation. Four colonies termed “LiLi” were chosen to inoculate four tubes of 2 mL liquid LB media with of erythromycin. A plasmid extraction was performed using the Qiagen MiniPrep plasmid extraction kit, yielding approximately 30 ng/μL of DNA per colony. FIG. 4 compares the sizes of several extracted plasmids to a ladder and pTRKH2.

As demonstrated by FIG. 4, LiLi5, LiLi7, and LiLi3 were larger plasmids than pTRKH2, suggesting that they may have been the product of a successful ligation reaction between the pTRKH2 backbone and the human PAH cDNA. LiLi6 appeared to be simply a re-ligation of the pTRKH2 backbone. LiLi8 was an uncharacterized plasmid. Subsequent digests revealed that LiLi3 lacked appropriate enzyme cut sites and was thus excluded from further experimentation.

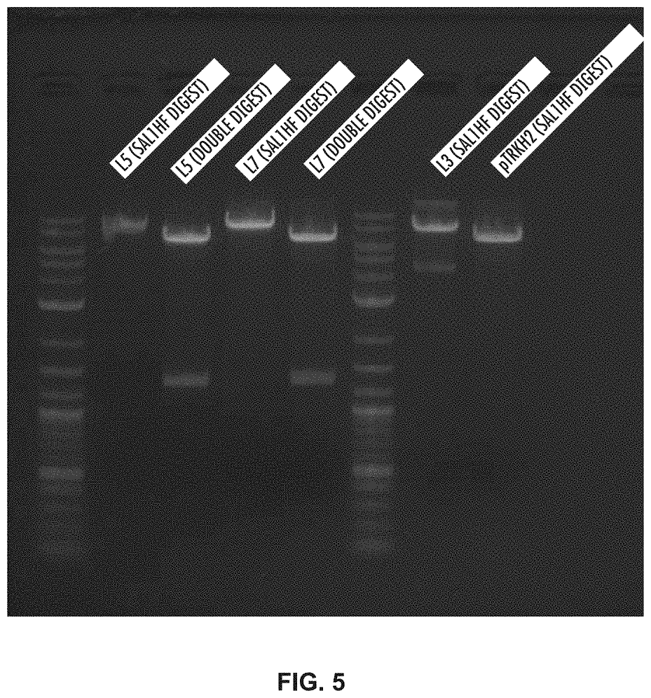

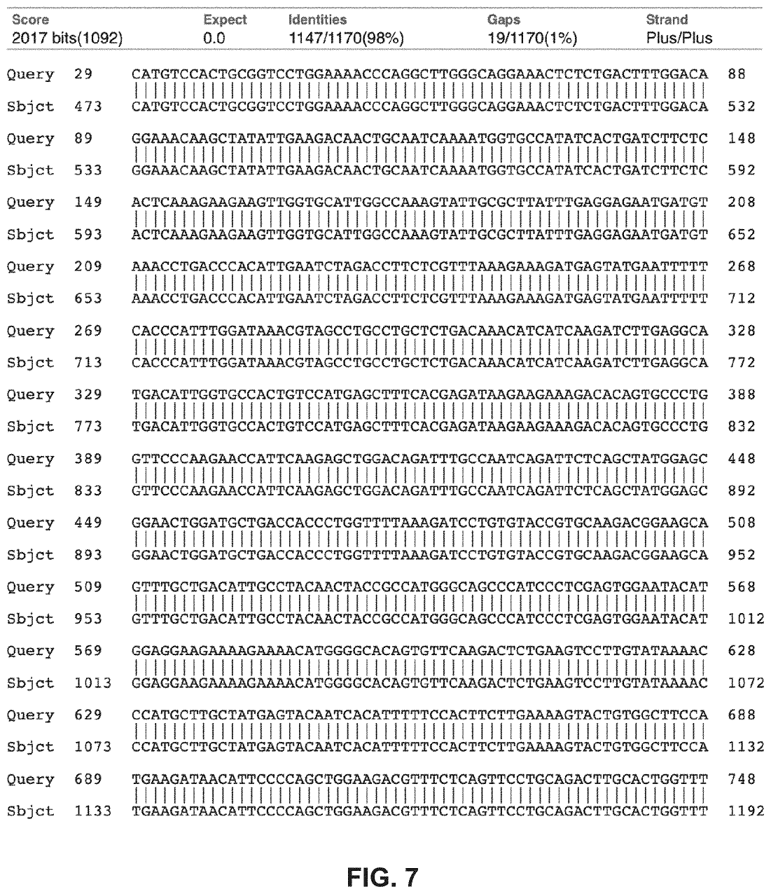

The plasmids LiLi5 and LiLi7 were then each digested in two separate reactions. A single digest with Sal1 and a double digest with Sal1 and Sac1 were conducted in the manner described above. The single digest linearized the plasmids to determine approximate size and the presence of appropriate cut sites in the plasmid. The left lanes of FIG. 5 show the results of the single and double digests, respectively. Double digestion revealed that the 1.3 kb PAH insert was removed from pTRKH2 backbone, validating that LiLi5 and LiLi7 contained a DNA fragment of the correct size and was correctly excised from plasmid with the restriction enzymes corresponding to the ligation sites. LiLi5 and LiLi7 were then sent for Sanger sequencing. The read length for LiLi7 was deemed too short to be acceptable and was not further analyzed. The LiLi5 sequence (SEQ ID NO: 3) was aligned with the NCBI sequence for the PAH (NM_000277.2) (the aligned portion of NM_000277.2 is shown in SEQ ID NO: 6) and determined to be 100% identical across the first 716 bases aligned (FIG. 7) and 99.8% identical across the first 994 bases aligned (data not shown), which is typically the upper limit of accuracy for standard Sanger sequencing.

Both the LiLi5 and pTRKH2 plasmids were transfected into Lactobacillus helveticus in the manner described in Materials and Methods. pTRKH2 was carried through the electroporation as a control to demonstrate positive selection for Lactobacillus helveticus containing plasmids yielding erythromycin resistance in liquid culture with antibiotic selection. The successful transfection with pTRKH2 and LiLi5 further validated electroporation protocols and characterized the behavior of Lactobacillus helveticus post-transfection with plasmid. After 96 hours, growth in liquid media was noted for cultures undergoing electroporation to introduce LiLi5 and pTRKH2 plasmids, suggesting that a plasmid conferring erythromycin resistance had successfully been transferred into Lactobacillus helveticus.

To confirm that the erythromycin resistance was a result of the LiLi5 and pTRKH2 plasmids being expressed in the Lactobacilli, a DNA extraction was performed using the ZymoPREP™ Plasmid MiniPrep Kit, per manufacturer protocol. A standard PCR reaction was then performed on the eluted DNA using primers specific to the PAH cDNA.

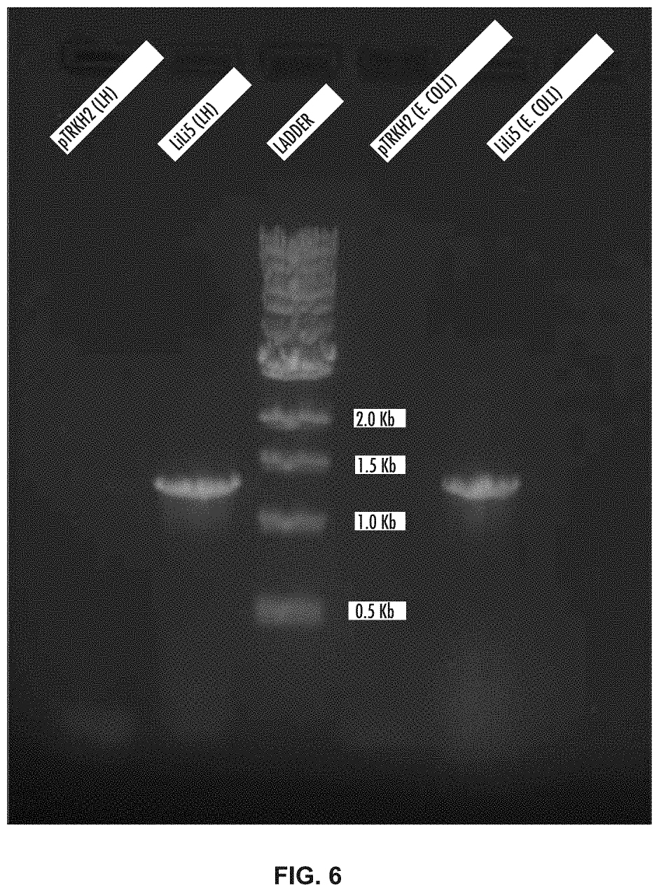

FIG. 6 shows the results of the PCR amplification used to confirm the presence of LiLi5 in Lactobacillus helveticus. The presence of a band at ˜1.3 kb, the identical size to the original cDNA isolated from pCMV6-XL4, confirms that the sequence encoding the human PAH enzyme was present in the modified Lactobacillus helveticus cells.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only and is not intended to limit the invention further described in the appended claims.

1. A modified bacterium comprising a bacterium having a genetic modification, wherein the genetic modification encodes an enzyme that encourages conversion of phenylalanine to tyrosine.

2. The modified bacterium of claim 1, wherein the genetic modification comprises a human cDNA sequence.

3. The modified bacterium of claim 1, wherein the genetic modification encodes SEQ ID NO: 1.

4. The modified bacterium of claim 1, wherein the human cDNA sequence comprises SEQ ID NO: 2, SEQ ID NO: 3, SEQ ID NO: 4, SEQ ID NO: 5, or SEQ ID NO: 6.

5. The modified bacterium of claim 1, wherein the bacterium is of the genus Lactobacillus.

6. The modified bacterium of claim 5, wherein the species is L. helveticus.

7. The modified bacterium of claim 1, wherein the modified bacterium survives in the human gut for at least 6 hours.

8. The modified bacterium of claim 1, further comprising a second genetic modification.

9. The modified bacterium of claim 7, wherein the second genetic modification encodes a signal sequence, an antibiotic resistance sequence, a control sequence, or a monitoring sequence.

10. An edible composition comprising the modified bacterium of any of claim 1.

11. A method of treating a metabolic disorder comprising delivering the modified bacterium of claim 1 to a patient.

12. The method of treating a metabolic disorder of claim 11, wherein the administration route is selected from one or more of the group consisting of: oral, rectal, nasogastric, percutaneous gastronomy endoscopy (PEG), nasoduodenal, and jejunostomy.

13. The method of treating a metabolic disorder of claim 11, wherein the metabolic disorder comprises phenylketonuria.

14. The method of treating a metabolic disorder of claim 11, wherein the patient is a pregnant mother or a child.

15. The method of treating a metabolic disorder of claim 11, further comprising delivering a secondary treatment to the patient.

16. The method of claim 15, wherein the secondary treatment comprises a pterin cofactor.

17. The method of claim 16, wherein the pterin cofactor comprises tetrahydrobiopterin.

18. The edible composition of claim 10, wherein the edible composition comprises a yogurt.


SEQUENCE LISTING (11 sequences)

SEQ ID NO: 1 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 2 - DNA, 1379 bp, Homo sapiens
cgcggagctcatgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaagtcgacgcgc

SEQ ID NO: 3 - DNA, 1369 bp, Homo sapiens
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaagtcgacgcgc

SEQ ID NO: 4 - DNA, 720 bp, Homo sapiens
catgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggttt

SEQ ID NO: 5 - DNA, 720 bp, Homo sapiens
catgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggttt

SEQ ID NO: 6 - DNA, 1370 bp, Homo sapiens
catgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaaagccatggac

SEQ ID NO: 7 - DNA, 33 bp, Artificial Sequence
  Description of Artificial Sequence Synthetic primer
atgtccactgcggtcctggaaaacccaggcttg

SEQ ID NO: 8 - DNA, 36 bp, Artificial Sequence
  Description of Artificial Sequence Synthetic primer
ttactttattttctggagggcactgcaaaggattcc

SEQ ID NO: 9 - DNA, 43 bp, Artificial Sequence
  Description of Artificial Sequence Synthetic primer
cgcggagctcatgtccactgcggtcctggaaaacccaggcttg

SEQ ID NO: 10 - DNA, 46 bp, Artificial Sequence
  Description of Artificial Sequence Synthetic primer
gcgcgtcgacttactttattttctggagggcactgcaaaggattcc

SEQ ID NO: 11 - DNA, 8060 bp, Artificial Sequence
  Description of Artificial Sequence Synthetic polynucleotide
ccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcatcgatatctagatctcgagctcatgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaagtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagaatcgatacgattttgaagtggcaacagataaaaaaaagcagtttaaaattgttgctgaacttttaaaacaagcaaatacaatcattgtcgcaacagatagcgacagagaaggcgaaaacattgcctggtcgatcattcataaagcaaatgccttttctaaagataaaacgtataaaagactatggatcaatagtttagaaaaagatgtgatccgtagcggttttcaaaatttgcaaccaggaatgaattactatcccttttatcaagaagcgcaaaagaaaaacgaaatgatacaccaatcagtgcaaaaaaagatataatgggagataagacggttcgtgttcgtgctgacttgcaccatatcataaaaatcgaaacagcaaagaatggcggaaacgtaaaagaagttatggaaataagacttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaaggagctaaagaggtccctagcgctcttatcatggggaagctcggatcatatgcaagacaaaataaactcgcaacagcacttggagaaatgggacgaatcgagaaaaccctctttacgctggattacatatctaataaagccgtaaggagacgggttcaaaaaggtttaaataaaggagaagcaatcaatgcattagctagaactatattttttggacaacgtggagaatttagagaacgtgctctccaagaccagttacaaagagctagtgcactaaacataattattaacgctataagtgtgtggaacactgtatatatggaaaaagccgtagaagaattaaaagcaagaggagaatttagagaagatttaatgccatatgcgtggccgttaggatgggaacatatcaattttcttggagaatacaaatttgaaggattacatgacactgggcaaatgaatttacgtcctttacgtataaaagagccgttttattcttaatataacggctctttttatagaaaaaatccttagcgtggtttttttccgaaatgctggcggtaccccaagaattagaaatgagtagatcaaattattcacgaatagaatcaggaaaatcagatccaaccataaaaacactagaacaaattgcaaagttaactaactcaacgctagtagtggatttaatcccaaatgagccaacagaaccagagccagaaacagaatcagaacaagtaacattggatttagaaatggaagaagaaaaaagcaatgacttcgtgtgaataatgcacgaaatcgttgcttatttttttttaaaagcggtatactagatataacgaaacaacgaactgaatagaaacgaaaaaagagccatgacacatttataaaatgtttgacgacattttataaatgcatagcccgataagattgccaaaccaacgcttatcagttagtcagatgaactcttccctcgtaagaagttatttaattaactttgtttgaagacggtatataaccgtactatcattatatagggaaatcagagagttttcaagtatctaagctactgaatttaagaattgttaagcaatcaatcggaaatcgtttgattgctttttttgtattcatttatagaaggtggagtttgtatgaatcatgatgaatgtaaaacttatataaaaaatagtttattggagataagaaaattagcaaatatctatacactagaaacgtttaagaaagagttagaaaagagaaatatctacttagaaacaaaatcagataagtatttttcttcggagggggaagattatatatataagttaatagaaaataacaaaataatttattcgattagtggaaaaaaattgacttataaaggaaaaaaatctttttcaaaacatgcaatattgaaacagttgaatgaaaaagcaaaccaagttaattaaacaacctattttataggatttataggaaaggagaacagctgaatgaatatcccttttgttgtagaaactgtgcttcatgacggcttgttaaagtacaaatttaaaaatagtaaaattcgctcaatcactaccaagccaggtaaaagcaaaggggctatttttgcgtatcgctcaaaatcaagcatgattggcggtcgtggtgttgttctgacttccgaggaagcgattcaagaaaatcaagatacatttacacattggacacccaacgtttatcgttatggaacgtatgcagacgaaaaccgttcatacacgaaaggacattctgaaaacaatttaagacaaatcaataccttctttattgattttgatattcacacggcaaaagaaactatttcagcaagcgatattttaacaaccgctattgatttaggttttatgcctactatgattatcaaatctgataaaggttatcaagcatattttgttttagaaacgccagtctatgtgacttcaaaatcagaatttaaatctgtcaaagcagccaaaataatttcgcaaaatatccgagaatattttggaaagtctttgccagttgatctaacgtgtaatcattttggtattgctcgcataccaagaacggacaatgtagaattttttgatcctaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttaatagggcgtaataacgtcatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagtgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaagtaaaaaaagaagaaaaagaaagctatataaaggcgctgacaaattcttttgacttagagcatacattcattcaagagactttaaacaagctagcagaacgccctaaaacggacacacaactcgatttgtttagctatgatacaggctgaaaataaaacccgcactatgccattacatttatatctatgatacgtgtttgttttttctttgctgtttagcgaatgattagcagaaatatacagagtaagattttaattaattattagggggagaaggagagagtagcccgaaaacttttagttggcttggactgaacgaagtgagggaaaggctactaaaacgtcgaggggcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgattacatggattggattagttcttgtggttacgtggtttttaactaaaagtagtgaatttttgatttttggtgtgtgtgtcttgttgttagtatttgctagtcaaagtgattaaatagaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctag